SEQUENCE LISTING (59 sequences)

SEQ ID NO: 1 - protein, 401 aa, Mycobacterium vaccae
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 2 - protein, 364 aa, Candida boidinii
MKIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 3 - DNA, 1206 bp, Mycobacterium vaccae
atggctaaggtcctgtgcgttctttacgatgatccagttgacggctaccctaagacctacgcccgcgacgatcttccaaagatcgaccactaccctggcggccagatcctcccaaccccaaaggccatcgacttcacccctggccagctcctcggctccgtctccggcgaactcggcctgcgcgaatacctcgaatccaacggccacaccctggtcgttacctccgacaaggacggcccagactccgttttcgagcgcgagctggtcgatgcagatgtcgtcatctcccagccattctggccagcctacctgaccccagagcgcatcgccaaggctaagaacctgaagctcgctctcaccgctggcatcggttccgaccacgtcgatcttcagtccgctatcgaccgcaacgtcaccgttgcagaagtcacctactgcaactccatcagcgtcgccgagcacgtggttatgatgatcctgtccctggttcgcaactacctgccttcccacgaatgggcgcgcaagggcggctggaacatcgccgactgcgtctcccacgcctacgacctcgaagctatgcacgtcggcaccgttgctgccggccgcatcggtctcgcagttctgcgccgtctggcaccattcgacgttcacctgcactacaccgaccgtcaccgcctgcctgaatccgtcgagaaggaactcaacctcacctggcacgcaacccgcgaggacatgtacccagtttgcgacgtggttaccctgaactgcccactgcacccagaaaccgagcacatgatcaatgacgagaccctgaagctgttcaagcgtggcgcctacatcgtcaacaccgcacgcggcaagctgtgcgaccgcgatgctgttgcacgtgctctcgaatccggccgcctggccggctacgccggcgacgtttggttcccacagcctgcaccaaaggaccacccatggcgcaccatgccatacaacggcatgaccccacacatctccggcaccaccctgaccgcacaggcacgttacgcagcaggcacccgcgagatcctggagtgcttcttcgagggccgtcctatccgcgacgaatacctcatcgttcagggcggcgctcttgctggcaccggcgcacattcctactccaagggcaatgccaccggcggttccgaagaggccgctaagttcaagaaggcagtctga

SEQ ID NO: 4 - DNA, 1095 bp, Candida boidinii
atgaaaatcgtgctggtattgtacgatgctggcaaacacgctgcagacgaggaaaagctctacggatgcacagaaaacaagctcggaattgcgaactggttgaaggaccagggtcatgaattgatcacaacttccgacaaggagggcggcaactctgtgctggatcaacatatcccggatgccgacattatcatcacgaccccatttcaccccgcatacatcaccaaggaacgcatcgataaagccaaaaagctgaagttggttgtggtcgctggtgttggctcagaccatattgatcttgattacatcaaccagacgggcaaaaagatttccgtgctggaagtgaccggttccaacgtcgtttctgtcgctgagcacgtcgttatgaccatgttggtcctggttcgtaatttcgttcccgcacacgagcagattattaaccatgactgggaagttgctgccatcgctaaggacgcttacgacattgagggtaagactattgccactatcggagccggacgcattggataccgcgtccttgaacgtctggtaccgttcaacccaaaggaactgctctactatgactaccaagccctccccaaagatgcagaggagaaagtcggtgcccgtcgcgtggagaatatcgaagagctcgtcgcacaggcagatattgtgacggtgaacgcaccattgcacgcgggaaccaagggcttgatcaacaaagagcttctcagcaagtttaagaaaggcgcatggctggtgaacactgcgcgcggagcaatctgtgtggcagaagatgttgcggctgccctggaatcgggtcagcttcgaggttatggcggcgatgtatggttccctcaacctgcgcctaaagaccacccatggcgagatatgcgtaataaatatggcgcgggtaacgctatgaccccacactactccggtaccacccttgatgcgcagacccgctatgcgcaaggcacaaagaatatcctcgaaagcttctttaccggcaagttcgattaccgcccgcaggacatcatcctcctgaatggcgaatacgtgaccaaggcctatggcaagcacgataaaaagtaa

SEQ ID NO: 5 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 6 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 7 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 8 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 9 - protein, 250 aa, Corynebacterium glutamicum
MSHMINKSISSTAEAVADIPDGASIAVGGFGLVGIPTALILALREQGAGDLTIISNNLGTDGFGLGLLLLDKKISKSIGSYLGSNKEYARQYLEGELTVEFTPQGTLAERLRAGGAGIPAFYTTAGVGTQVAEGGLPQRYNTDGTVAVVSQPKETREFNGQLYVMEEGIRADYALVHAHKADRFGNLVFRKTAQNFNPDAAMSGKITIAQVEHFVDELHPDEIDLPGIYVNRVVHVGPQETGIENRTVSN

SEQ ID NO: 10 - DNA, 43 bp, Artificial Sequence
  Synthetic (ldhA_5′_HindIII)
catgattacgccaagcttgagagcccaccacattgcgatttcc

SEQ ID NO: 11 - DNA, 42 bp, Artificial Sequence
  Synthetic (ldhA_up_3′_XhoI)
tcgaaactcgagtttcgatcccacttcctgatttccctaacc

SEQ ID NO: 12 - DNA, 39 bp, Artificial Sequence
  Synthetic (ldhA_dn_5′_XhoI)
tcgaaactcgagtaaatctttggcgcctagttggcgacg

SEQ ID NO: 13 - DNA, 46 bp, Artificial Sequence
  Synthetic (ldhA_3′_EcoRI)
acgacggccagtgaattcgacgacatctgagggtggataaagtggg

SEQ ID NO: 14 - DNA, 42 bp, Artificial Sequence
  Synthetic (poxB 5′ H3)
catgattacgccaagctttcagcgtgggtcgggttctttgag

SEQ ID NO: 15 - DNA, 32 bp, Artificial Sequence
  Synthetic (DpoxB_up 3′)
aatcatcatctgaactcctcaacgttatggct

SEQ ID NO: 16 - DNA, 37 bp, Artificial Sequence
  Synthetic (DpoxB_dn 5′)
ggagttcagatgatgattgatacacctgctgttctca

SEQ ID NO: 17 - DNA, 44 bp, Artificial Sequence
  Synthetic (poxB 3′ E1)
acgacggccagtgaattcatgtcccgaatccacttcaatcagag

SEQ ID NO: 18 - DNA, 44 bp, Artificial Sequence
  Synthetic (pta 5′ H3)
catgattacgccaagcttccctccatgatacgtggtaagtgcag

SEQ ID NO: 19 - DNA, 43 bp, Artificial Sequence
  Synthetic (Dpta_up_R1 3′)
gttccctgttaatgtaaccagctgaggtcggtgtgtcagacat

SEQ ID NO: 20 - DNA, 48 bp, Artificial Sequence
  Synthetic (DackA_dn_R1 5′)
ttacattaacagggaaccggaagagttagctatcgctaggtacgcggt

SEQ ID NO: 21 - DNA, 40 bp, Artificial Sequence
  Synthetic (ackA 3′ Xb)
acccggggatcctctagagggctgatgtgatttctgcggg

SEQ ID NO: 22 - DNA, 41 bp, Artificial Sequence
  Synthetic (actA 5′ Xb)
ggtggcggccgctctagaggtctgagctttattcctgggct

SEQ ID NO: 23 - DNA, 36 bp, Artificial Sequence
  Synthetic (DactA_up_R4 3′)
tctggatagaagcatctaagccagcgccggtgaagc

SEQ ID NO: 24 - DNA, 46 bp, Artificial Sequence
  Synthetic (DactA_dn_R4 5′)
agatgcttctatccagagctccggtgacaacaagtacatgcagacc

SEQ ID NO: 25 - DNA, 39 bp, Artificial Sequence
  Synthetic (actA 3′ H3)
gacggtatcgataagcttcgtacgatgcttgagcggtat

SEQ ID NO: 26 - DNA, 19 bp, Artificial Sequence
  Synthetic (poxB_up_for)
ggctgaaaccaaaccagac

SEQ ID NO: 27 - DNA, 22 bp, Artificial Sequence
  Synthetic (poxB_dn_rev)
ctgcatgatcggttagatacag

SEQ ID NO: 28 - DNA, 18 bp, Artificial Sequence
  Synthetic (pta_up_for)
gcgtggaattgagatcgg

SEQ ID NO: 29 - DNA, 18 bp, Artificial Sequence
  Synthetic (ackA_dn_rev)
cagagcgatttgtggtgg

SEQ ID NO: 30 - DNA, 20 bp, Artificial Sequence
  Synthetic (actA_up_for)
tgaagcaatggtgtgaactg

SEQ ID NO: 31 - DNA, 19 bp, Artificial Sequence
  Synthetic (actA_dn_rev)
gctaccaaacactagcctg

SEQ ID NO: 32 - DNA, 46 bp, Artificial Sequence
  Synthetic (MD-616)
aaagtgtaaagcctgggaacaacaagacccatcatagtttgccccc

SEQ ID NO: 33 - DNA, 36 bp, Artificial Sequence
  Synthetic (MD-618)
gttcttctaatcagaattggttaattggttgtaaca

SEQ ID NO: 34 - DNA, 40 bp, Artificial Sequence
  Synthetic (MD-615)
gcgtaatagcgaagaggggcgtttttccataggctccgcc

SEQ ID NO: 35 - DNA, 40 bp, Artificial Sequence
  Synthetic (MD-617)
gttcaatcataacaccccttgtattactgtttatgtaagc

SEQ ID NO: 36 - DNA, 31 bp, Artificial Sequence
  Synthetic (MD-619)
gggtgttatgattgaacaagatggattgcac

SEQ ID NO: 37 - DNA, 39 bp, Artificial Sequence
  Synthetic (MD-620)
attctgattagaagaactcgtcaagaaggcgatagaagg

SEQ ID NO: 38 - DNA, 17 bp, Artificial Sequence
  Synthetic (LacZa-NR)
cctcttcgctattacgc

SEQ ID NO: 39 - DNA, 21 bp, Artificial Sequence
  Synthetic (MD-404)
cccaggctttacactttatgc

SEQ ID NO: 40 - DNA, 47 bp, Artificial Sequence
  Synthetic (MD-627)
gccaccgcggtggagctcatttagcggatgattctcgttcaacttcg

SEQ ID NO: 41 - DNA, 32 bp, Artificial Sequence
  Synthetic (MD-628)
ttttatttgcaaaaacggccgaaaccatccct

SEQ ID NO: 42 - DNA, 40 bp, Artificial Sequence
  Synthetic (MD-629)
ccgtttttgcaaataaaacgaaaggctcagtcgaaagact

SEQ ID NO: 43 - DNA, 43 bp, Artificial Sequence
  Synthetic (MD-630)
gaacaaaagctggagctaccgtatctgtggggggatggcttgt

SEQ ID NO: 44 - DNA, 36 bp, Artificial Sequence
  Synthetic (Tuf-F)
ctatagggcgaattgggatcacagtaggcgcgtagg

SEQ ID NO: 45 - DNA, 56 bp, Artificial Sequence
  Synthetic (Tuf-R)
gacctcgagggggggcccggtaccggttgtcctcctttgggtggctacgactttcg

SEQ ID NO: 46 - DNA, 50 bp, Artificial Sequence
  Synthetic (J0180)
ctatagggcgaattgggtacctgcgttaataaaggtggagaataagttgt

SEQ ID NO: 47 - DNA, 41 bp, Artificial Sequence
  Synthetic (MD-1081)
tgacctcctctcgagtttagattccctaaacttttatcgag

SEQ ID NO: 48 - DNA, 50 bp, Artificial Sequence
  Synthetic (Mv_fdh_5′_F)
gggaatctaaactcgaggaaggagatatacatatggctaaggtcctgtgc

SEQ ID NO: 49 - DNA, 37 bp, Artificial Sequence
  Synthetic (Mv_fdh_3′_R)
catccgctaaatgagctctcagactgccttcttgaac

SEQ ID NO: 50 - DNA, 50 bp, Artificial Sequence
  Synthetic (Cb_fdh_5′_F)
gggaatctaaactcgaggaaggagatatacatatgaaaatcgtgctggta

SEQ ID NO: 51 - DNA, 37 bp, Artificial Sequence
  Synthetic (Cb_fdh_3′_R)
catccgctaaatgagctcttactttttatcgtgcttg

SEQ ID NO: 52 - DNA, 29 bp, Artificial Sequence
  Synthetic (poxB_up_NF)
gattacgccaagctttcagcgtgggtcgg

SEQ ID NO: 53 - DNA, 31 bp, Artificial Sequence
  Synthetic (poxB_up_NR)
attaacgcagctgaactcctcaacgttatgg

SEQ ID NO: 54 - DNA, 36 bp, Artificial Sequence
  Synthetic (poxB_dn_NF)
ccacagatacggtatgatgattgatacacctgctgt

SEQ ID NO: 55 - DNA, 36 bp, Artificial Sequence
  Synthetic (poxB_dn_NR)
acggccagtgaattcatgtcccgaatccacttcaat

SEQ ID NO: 56 - DNA, 36 bp, Artificial Sequence
  Synthetic (poxB_fdh_NF)
aggagttcagctgcgttaataaaggtggagaataag

SEQ ID NO: 57 - DNA, 28 bp, Artificial Sequence
  Synthetic (poxB_fdh_NR)
atcaatcatcataccgtatctgtggggg

SEQ ID NO: 58 - DNA, 24 bp, Artificial Sequence
  Synthetic (poxB_C_F)
gaagtcatggatcgtaactgtaac

SEQ ID NO: 59 - DNA, 21 bp, Artificial Sequence
  Synthetic (poxB_C_R)
gttgtttagagcctgaagctc

(19) United States
(12) Patent Application Publication
(10) Pub. No.: US 2015/0064753 A1
(43) Pub. Date: Mar. 5, 2015

(54) CORYNEBACTERIUM COMPRISING NAD+ DEPENDENT FORMATE DEHYDROGENASE GENE AND METHOD FOR PRODUCING C4 DICARBOXYLIC ACID USING THE SAME

(71) Applicant: Samsung Electronics Co., Ltd., Suwon-si (KR)

(72) Inventors: Soonchun CHUNG, Seoul (KR); Joonsong PARK, Seoul (KR); Jinhwan PARK, Suwon-si (KR); Jiae YUN, Hwaseongi-si (KR); Jaechan PARK, Yongin-si (KR); Kwangmyung CHO, Seongnam-si (KR)

(21) Appl. No.: 14/478,628

(22) Filed: Sep. 5, 2014

(30) Foreign Application Priority Data
Sep. 5, 2013 (KR) 10-2013-0106818

(51) Int. Cl. C12P 7/46; C12N 15/77; C12N 9/02

(52) U.S. Cl. CPC C12P 7/46; C12N 9/0008; C12N 15/77; C12Y 102/01002
USPC 435/145; 435/471; 435/252.32

(57) ABSTRACT

A Corynebacterium including an NAD+ dependent formate dehydrogenase gene, and a method of producing C4 dicarboxylic acid using the Corynebacterium.

### CROSS-REFERENCE TO RELATED APPLICATIONS

This application claims the benefit of Korean Patent Application No. 10-2013-0106818, filed on Sep. 5, 2013 in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 37,448 bytes ASCII (Text) file named “718133_ST25.TXT,” created Sep. 4, 2014.

### BACKGROUND

1. Field

The present disclosure relates to Corynebacterium including a gene that encodes NAD+ dependent formate dehydrogenase and methods of producing C4 dicarboxylic acids using the Corynebacterium.

2. Description of the Related Art

Microorganisms of Corynebacterium are gram positive strains, which are widely used for producing amino acids such as glutamate, lysine, and threonine. Corynebacterium glutamicum has simple growth conditions, stable genomic structure, and is free of environmental hazards. Thus, Corynebacterium glutamicum has advantages as a commercial strain.

Corynebacterium glutamicum is an aerobic bacterium, which produces lactic acid, acetic acid, succinic acid and the like under anaerobic conditions in order to produce the minimal energy required for survival under conditions in which oxygen supply is insufficient or absent. When Corynebacterium undergoes a reductive tricarboxylic acid (TCA) cycle under anaerobic conditions, oxalacetic acid is converted into malic acid, which is then converted into fumaric acid, which is then converted into succinic acid. Two moles of NADH are required during this process.

NAD+ dependent formate dehydrogenase is an enzyme that catalyzes oxidation of formate into bicarbonate or CO₂. The enzyme may donate electrons to NAD+ to catalyze the production of NADH. The enzyme increases the amount of NADH in cells to create an advantageous environment for producing reductive metabolites.

However, it is known that Corynebacterium does not have an enzyme such as NAD+ dependent formate dehydrogenase. Hence, a method of using Corynebacterium under anaerobic conditions to increase the production of reductive metabolites is needed.

### SUMMARY

Provided is a recombinant Corynebacterium microorganism comprising a gene that encodes NAD+ dependent formate dehydrogenase (FDH).

Also provided is a method of producing a C4 dicarboxylic acid comprising culturing the recombinant Corynebacterium microorganism to produce a cultured product comprising a C4 dicarboxylic acid; and recovering the C4 dicarboxylic acid from the cultured product.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a map of a pGSK+ vector;

FIG. 2 is a map of a pGST1 vector;

FIG. 3 is a map of a pGS-EX4 vector;

FIG. 4A is a graph showing changes in the amount of glucose consumption plotted against culture time of Corynebacterium with and without an fdh gene expression strain, before and after adding formate.

FIG. 4B is a graph showing inhibitory effects of formate on the production of succinic acid;

FIG. 5 is a map of a pK19ms_ΔpoxB_P29::Mv.fdh vector; and

FIG. 6A is a graph showing succinate productivity of a strain including a genomically integrated fdh gene compared to a parent strain.

FIG. 6B is a graph showing the glucose consumption rate of a strain including a genomically integrated fdh gene compared to a parent strain.

FIG. 6C is a graph showing the succinic acid yield of a strain including a genomically integrated fdh gene compared to a parent strain.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Provided are microorganisms of Corynebacterium including a gene encoding NAD+ dependent formate dehydrogenase (FDH).

The gene may be, for example, derived from bacteria or yeast. The gene may be derived from, for example, Pseudomonas sp., Moraxella sp., Paracoccus sp., Mycobacterium vaccae, or Hyphomicrobium sp. The gene may be derived from methylotrophic yeast such as Pichia angusta, Candida methylica, or Candida boidinii. Thus, the gene may be heterologous (non-native) to the Corynebacterium.

The FDH may be formate: NAD+ oxydoreductase in the category of EC.1.2.1.2. The FDH may have an amino acid sequence of SEQ ID NO: 1 or SEQ ID NO: 2.

The sequence of the gene encoding the FDH may be codon-optimised for use in Corynebacterium genus. The gene may have a nucleotide sequence of SEQ ID NO: 3 or SEQ ID NO: 4.

The gene may be inserted (or integrated) into a chromosome, or may not be inserted into a chromosome (e.g., may be introduced as part of a stable extra-chromosomal vector). The gene may be inserted via a vehicle such as a vector. The vector may include a control sequence operably linked to the gene and/or a homologous region. As used herein, the expression “operably linked” denotes a functional association between a nucleic acid expression control sequence and another nucleotide sequence, and as a result, the control sequence controls the transcription and/or translation of a gene. The control sequence may include a promoter, a terminator, a ribosome binding site, an enhancer, or a combination thereof. The promoter may be, for example, an NCgl1929 promoter, a tuf promoter, or a tac promoter. The terminator may be, for example, an rrnB terminator. A homologous region is a region recognized by a recombinase, such that the homologous region is cross-linked with the corresponding site of a chromosome. The homologous region may be located upstream and/or downstream of a polynucleotide that is to be integrated. The integration of the gene into the chromosome may occur through a homologous recombination. For example, the integration may occur by inserting the gene into a vector to prepare a recombinant vector and introducing the recombinant vector into a microorganism to induce integration of the gene into the chromosome through a homologous recombination.

The microorganism may exhibit increased expression of NAD+ dependent formate dehydrogenase compared to a non-recombinant microorganism of the same type. The term “non-recombinant microorganism of the same type” means a reference microorganism with regard to the subject modification. The reference microorganism refers to a wild-type microorganism or a parental microorganism. The parental microorganism refers to a microorganism that has not undergone a subject modification but is genetically identical to the recombinant microorganism except for the modification (e.g., which has not been modified to include an NAD+ dependent formate dehydrogenase gene), and thus serves as a reference microorganism for the modification. The microorganism may include an increased amount of NAD+ dependent formate dehydrogenase proteins as compared to a non-recombinant Corynebacterium (e.g., a bacterium of the same type, but which has not been modified to include an NAD+ dependent formate dehydrogenase gene). The microorganism may be, for instance, Corynebacterium glutamicum or Corynebacterium thermoaminogenes, Brevibacterium flavum, and Brevibacterium lactofermentum.

The microorganism may have an inhibited or blocked synthesis pathway for producing lactate from pyruvate. The microorganism may have eliminated or reduced activity of L-lactate dehydrogenase (LDH). The microorganism may have inactivated or attenuated form of the gene encoding LDH. The LDH may be an enzyme categorized as EC.1.1.1.27. The LDH may have, for example, an amino acid sequence of SEQ ID NO: 5 or that having a sequence identity of about 70% or higher with SEQ ID NO: 5 (e.g., about 80% or higher, about 90% or higher, or about 95% or higher).

Also, the microorganism may have a inhibited or blocked synthesis pathway for producing acetate from pyruvate. The microorganism may have eliminated or reduced activity of at least one protein selected from the group consisting of pyruvate oxidase (PoxB), phosphotransacetylase (PTA), acetate kinase (AckA), and acetate coenzyme A transferase (ActA). The microorganism may have inactivated or attenuated form of at least one gene selected from the group consisting of a gene coding pyruvate oxydase, a gene coding phosphotransacetylase, a gene coding acetate kinase, and a gene coding acetate coenzyme A transferase. The PoxB may be an enzyme categorized as EC.1.2.5.1. The PoxB may have an amino acid sequence of SEQ ID NO: 6 or that having a sequence identity of about 70% or higher with SEQ ID NO: 6 (e.g., about 80% or higher, about 90% or higher, or about 95% or higher). The PTA may be an enzyme categorized as EC.2.3.1.8. The PTA may have an amino acid sequence of SEQ ID NO: 7 or that having a sequence identity of about 70% or higher with SEQ ID NO: 7 (e.g., about 80% or higher, about 90% or higher, or about 95% or higher). The AckA may be an enzyme categorized as EC.2.7.2.1. The AckA may have, for example, an amino acid sequence of SEQ ID NO: 8 or that having a sequence identity of about 70% or higher with SEQ ID NO: 7 (e.g., about 80% or higher, about 90% or higher, or about 95% or higher). The ActA may be an enzyme categorized as EC.2.8.3.8. The ActA may have an amino acid sequence of SEQ ID NO: 9 or that having a sequence identity of about 70% or higher with SEQ ID NO: 9 (e.g., about 80% or higher, about 90% or higher, or about 95% or higher). As used herein, the term “gene” may include a region encoding a protein, or a region encoding a protein and a region controlling the expression thereof.

The term “reduction” may denote a comparison of activity of a protein within a manipulated strain to the activity of the protein within a reference strain. The reference strain refers to a wild-type strain or a parental strain. The parental strain refers to a strain that has not undergone a subject modification but is genetically identical except for the modification, and thus serves as a reference strain for the modification. The term “inactivation” as used herein may denote the production of a gene that is not expressed at all, or a gene by which the protein encoded is not active even if expressed. The term “attenuation” may denote the production of a gene that is expressed at a lower level compared to a reference strain, or a gene by which the protein encoded has reduced activity compared to a reference strain even if expressed. The inactivation or attenuation may occur through a homologous recombination. The inactivation or attenuation may occur by introducing vectors including portions of sequences of the genes into cells to transform the cells, culturing the cells such that the sequences may undergo homologous recombination with endogenous genes corresponding thereto, and then selecting homologously recombined cells by using selection markers.

According to another embodiment, provided is a method of producing C4 dicarboxylic acid including culturing a Corynebacterium microorganism including a gene encoding NAD+ dependent formate dehydrogenase so as to produce a C4 dicarboxylic acid; and recovering C4 dicarboxylic acids from the culture.

The Corynebacterium microorganism is as described herein.

The culturing may be performed using a suitable culture medium and culturing conditions known in the art. A culturing method may include a batch culture, a continuous culture, and a fed-batch culture, or a combination thereof.

The culture medium may include various carbon sources, nitrogen sources, and trace element components.

The carbon sources may include carbohydrates such as glucose, sucrose, lactose, fructose, maltose, starch, and cellulose; fats such as soybean oil, sunflower oil, castor oil, and coconut oil; fatty acids such as palmitic acid, stearic acid, and linoleic acid; alcohols such as glycerol and ethanol; and organic acids such as acetic acid; or a combination thereof. The culturing may occur by having glucose as a carbon source. The nitrogen sources may include organic nitrogen sources such as peptone, yeast extract, beef stock, malt extract, corn steep liquor (CSL), and soybean flour, and inorganic nitrogen sources such as urea, ammonium sulfate, ammonium chloride, ammonium phosphate, ammonium carbonate, and ammonium nitrate, or a combination thereof. The culture medium is a supply source of phosphorus, and may include potassium dihydrogen phosphate, dipotassium hydrogen phosphate, and corresponding metal salts such as sodium-containing salt, magnesium sulfate, and iron sulfate. Also, an amino acid, a vitamin, and a suitable precursor may be included in a culture medium. The culture medium or an individual component may be added to the culture solution by a batch method or a continuous method.

Also, compounds such as ammonium hydroxide, potassium hydroxide, ammonia, phosphoric acid, and sulfuric acid may be added to a microorganism culture medium through a suitable method to adjust pH of the culture medium. Also, antifoaming agents such as fatty acid polyglycol ester may be added during the culturing to inhibit the production of bubbles.

The microorganism may be cultured without the addition of formate.

The microorganism may be cultured under microaerobic conditions or anaerobic conditions. As used herein, the term “anaerobic conditions” refers to an environment devoid of oxygen. As used herein, the term “microaerobic conditions” when used in reference to a culture or growth condition is intended to mean that the dissolved oxygen concentration in the medium remains between 0 and about 10% of saturation for dissolved oxygen in liquid media. Microaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. The anaerobic conditions may be created by supplying carbon dioxide or nitrogen gas at a flow rate of about 0.1 vvm (aeration volume/medium volume/minute) to about 0.4 vvm, about 0.2 vvm to about 0.3 vvm, or about 0.25 vvm. A culturing temperature may be about 20° C. to about 45° C. or about 25° C. to about 40° C. A culturing period may be continued until the desired amount of desired C4 dicarboxylic acid has been reached.

The C4 dicarboxylic acid may be an acid or a salt thereof having four carbon atoms and two carboxyl groups. For example, the C4 dicarboxylic acid may be malic acid, fumaric acid, or succinic acid.

A recovery of the C4 dicarboxylic acid may be performed by a known separation and purification method in the art. The recovery may occur through centrifugation, ion-exchange chromatography, filtration, precipitation, or a combination thereof.

### EXAMPLE 1

Preparation of a Strain in which Lactate and Acetate Synthesis Pathways are Removed

(1) Preparation of a Replacement Vector

Genes for L-lactate dehydrogenase (ldh), pyruvate oxidase (poxB), phosphotransacetylase (pat), acetate kinase (ackA), and acetate CoA transferase (actA) of Corynebacterium glutamicum (C. glutamicum, CGL) ATCC 13032 were inactivated through a homologous recombination. As a vector for inactivating the genes, pK19 mobsacB (ATCC 87098) vector was used, and two homologous recombinant regions to be used for the homologous recombination were obtained by PCR amplification using the genomic DNA of CGL ATCC 13032 as a template DNA.

Two homologous regions for removing the ldh gene are upstream and downstream of the gene, which were obtained by PCR amplification using an IdhA_{—}5′_HindIII (SEQ ID NO: 10) and IdhA_up_{—}3′_XhoI (SEQ ID NO: 11) primer set, and an IdhA_dn_{—}5′_XhoI (SEQ ID NO: 12) and IdhA_{—}3′_EcoRI (SEQ ID NO: 13) primer set, respectively. The PCR amplification was performed by amplification at a temperature of 95° C. for 30 seconds, annealing at a temperature of 55° C. for 30 seconds, and elongation at a temperature of 72° C. for 30 seconds, and repeating the same 30 times. Hereinafter, all PCR amplifications were performed under the same conditions. The obtained amplification products were cloned in HindIII and EcoRI restriction sites of pK19 mobsacB vector to prepare a pK19_Δldh vector.

Two homologous regions for removing a poxB gene are upstream and downstream of the gene, which were obtained by PCR amplification using a poxB 5′ H3 (SEQ ID NO: 14) and DpoxB_up 3′ (SEQ ID NO: 15) primer set, and a DpoxB_dn 5′ (SEQ ID NO: 16) and poxB 3′ E1 (SEQ ID NO: 17) primer set, respectively. The obtained amplification products were cloned in HindIII and EcoRI restriction sites of pK19 mobsacB vector to prepare pK19_ΔpoxB vector.

Two homologous regions for removing a pat-ackA gene are upstream and downstream of the gene, which were obtained by PCR amplification using a pat 5′ H3 (SEQ ID NO: 18) and Dpta_up_R1 3′ (SEQ ID NO: 19) primer set, and a DackA_dn_R1 5′ (SEQ ID NO: 20) and ackA 3′ Xb (SEQ ID NO: 21) primer set, respectively. The obtained amplification product was cloned in HindIII and XbaI restriction sites of pK19 mobsacB vector to prepare pK19_Δpat_ackA vector.

Two homologous regions for removing the actA gene are upstream and downstream of the gene, which were obtained by PCR amplification using an actA 5′ Xb (SEQ ID NO: 22) and DactA_up_R4 3′ (SEQ ID NO: 23) primer set, and a DactA_dn_R4 5′ (SEQ ID NO: 24) and actA 3′ H3 (SEQ ID NO: 25) primer set, respectively. The obtained amplification products were cloned in XbaI and HindIII restriction sites of pK19 mobsacB vector to prepare pK19_ΔactA vector.

(2) Preparation of CGL (Δldh, ΔpoxB, Δpat-ackA, and ΔactA)

The substituted vectors described above were introduced together into C. glutamicum ATCC13032 through electroporation. The strain was streaked on an LBHIS agar plate including 25 ug/ml (micrograms per milliliter) of kanamycin and cultured at a temperature of 30° C. The LBHIS agar plate includes 25 g/L of Difco LB™ broth, 18.5 g/L of brain-heart infusion broth, 91 g/L of D-sorbitol, and 15 g/L of agar. Hereinafter, the composition of the LBHIS culture medium is as described above. A colony formed was cultured in a BHIS culture medium including 37 g/L of brain heart infusion powder, and 91 g/L of D-sorbitol (pH of 7.0) at a temperature of 30° C., and then the culture medium was streaked on an LB/Suc10 agar plate and then cultured at a temperature of 30° C., followed by selecting colonies in which double cross-linking has occurred. The LB/Suc10 agar plate includes 25 g/L of Difco LB™ broth, 15 g/L of agar, and 100 g/L of sucrose.

After isolating genomic DNA from selected colonies, the deletion of the genes was confirmed. The deletion of an ldh gene was confirmed by using an ldhA_{—}5′_HindIII and ldhA_{—}3′_EcoRI primer set, and the deletion of the poxB gene was confirmed through PCR by using a poxB_up_for (SEQ ID NO: 26) and poxB_dn_rev (SEQ ID NO: 27) primer set. Also, the deletion of the pat-ackA gene was confirmed through PCR by using a pat_up_for (SEQ ID NO: 28) and ackA_dn_rev (SEQ ID NO: 29) primer set, and the deletion of the actA gene was confirmed through PCR by using an actA_up_for (SEQ ID NO: 30) and actA_dn_rev (SEQ ID NO: 31) primer set.

### EXAMPLE 2

Preparation of NAD+ Dependent Formate Dehydrogenase (fdh) Gene Expression Strain and Confirmation Of Inhibitory Effects of Formate on Succinic Acid Production

(1) Preparation of pGEX_Ptuf::Mv.fdh and pGEX_Ptuf::Cb.fdh Vector

1) Preparation of pGS EX4 Vector

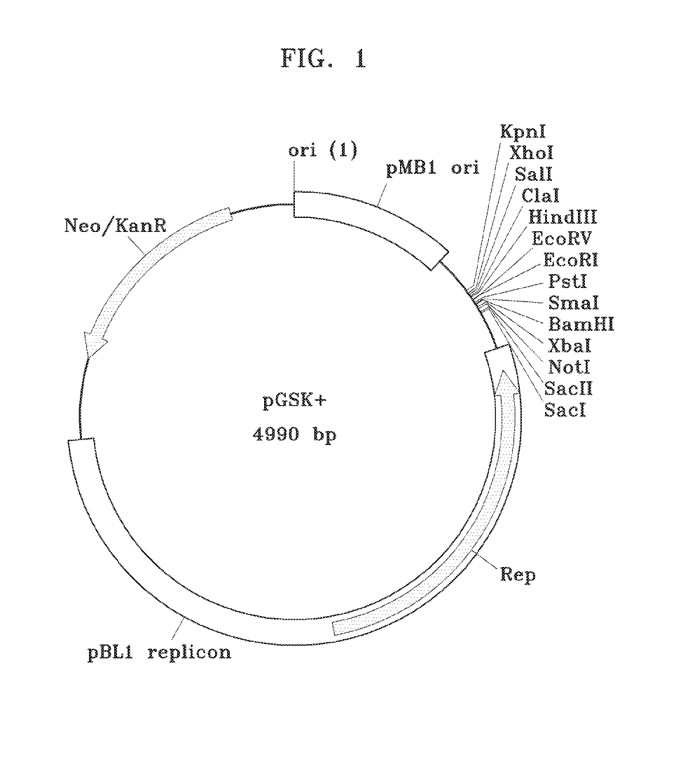

The following four PCR products were obtained by using Phusion High-Fidelity DNA Polymerase (cat.# M0530, available from New England Biolabs). PCR was performed by using pET2 (GenBank accession number: AJ885178.1), which is a vector for promoter screening of Corynebacterium glutamicum, as a template, along with an MD-616 (SEQ ID NO: 32) and MD-618 (SEQ ID NO: 33) primer set, and an MD-615 (SEQ ID NO: 34) and MD-617 (SEQ ID NO: 35) primer set. Also, PCR was performed by using pEGFP-C1 (available from Clontech) as a template and an MD-619 (SEQ ID NO: 36) and MD-620 (SEQ ID NO: 37) primer set, and PCR was performed by using pBluescriptll SK+ as a template and an LacZa-NR (SEQ ID NO: 38) and MD-404 (SEQ ID NO: 39) primer set. Each of 3010 bp, 854 bp, 809 bp, and 385 by fragments, which are PCR products, were cloned into a circular plasmid according to a method described in In-Fusion EcoDry PCR cloning kit (cat.# 639690, available from Clontech). Cloned vectors were introduced into One Shot TOP10 chemically competent cells (cat.# C4040-06, available from Invitrogen), cultured in an LB culture medium including 25 mg/L of kanamycin, and then growing colonies were selected. A vector was recovered from selected colonies to confirm vector sequences through sequencing. The vector was named pGSK+ (FIG. 1).

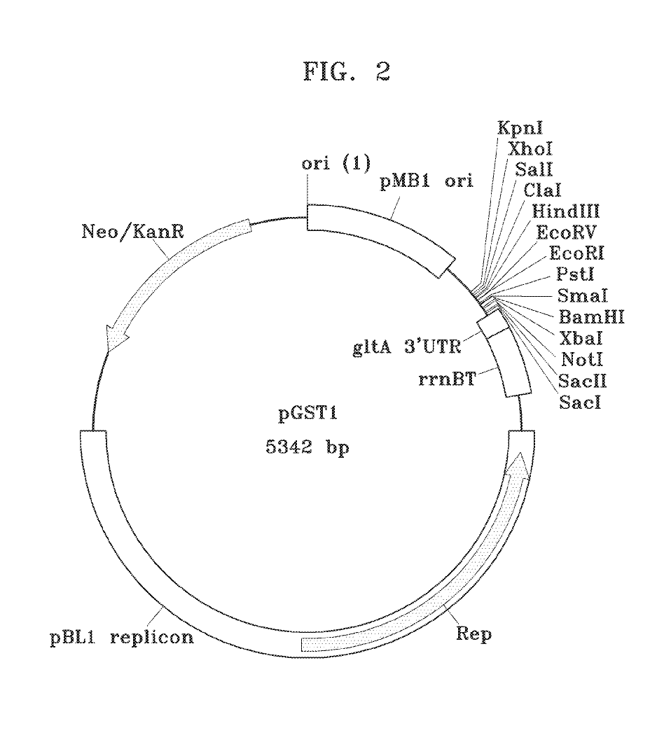

Also, 3′UTR of C. glutamicum gltA (NCgl 0795) and rho-independent terminator of E. coli rrnB were inserted into the pGSK+ vector as follows. PCR was performed by using the genomic DNA of C. glutamicum (ATCC13032) as a template and an MD-627 (SEQ ID NO: 40) and MD-628 (SEQ ID NO: 41) primer set, to obtain a 108 by PCR fragment of 3′UTR of gltA. Also, a 292 by PCR product of rrnB transcription terminator was obtained by using E. coli (MG1655) genomic DNA as a template and an MD-629 (SEQ ID NO: 42) and MD-630 (SEQ ID NO: 43) primer set. Two of the amplified fragments described above were inserted into the pGSK+ vector which was cut by SacI, by using an In-Fusion EcoDry PCR cloning kit (cat.# 639690, available from Clontech). Cloned vectors were introduced into One Shot TOP10 chemically competent cells (cat.# C4040-06, available from Invitrogen), cultured in an LB culture medium including 25 mg/L of kanamycin and then growing colonies were selected. Vectors were recovered from the selected colonies to confirm vector sequences through sequencing. The vector was named pGST1 (FIG. 2).

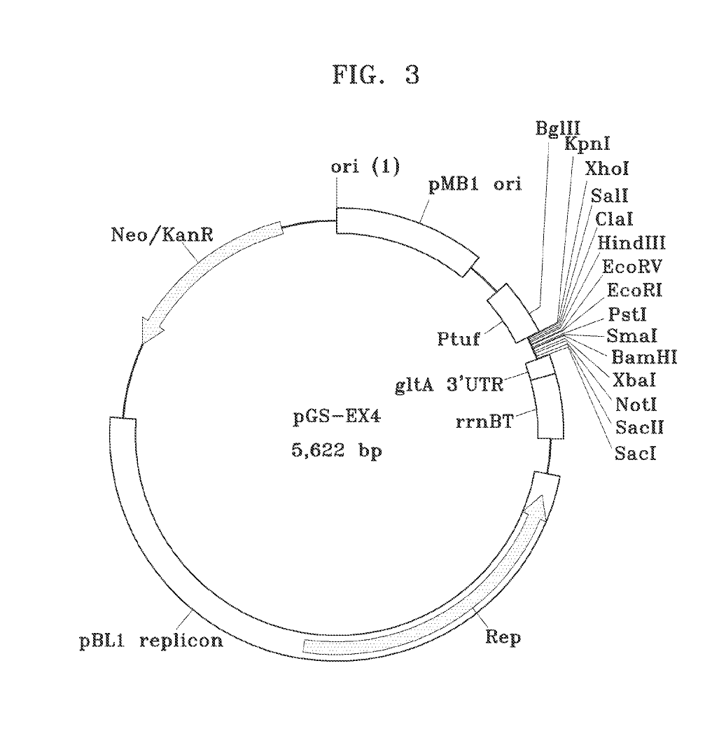

Also, Ptuf fragments were obtained by using the genomic DNA of C. glutamicum ATCC 13032 as a template, and a Tuf-F (SEQ ID NO: 44) and Tuf-R (SEQ ID NO: 45) primer set. Ptuf is a promoter of a tuf gene (NCgl 0480) derived from Corynebacterium glutamicum. The obtained Ptuf fragments were cloned in a KpnI site of the pGST1 vector by using an In-Fusion® HD cloning kit (639648, available from Clontech) to obtain pGS_EX4 vector (FIG. 3).

2) Preparation of pDGEX Ptuf::Mv.fdh and pGEX Ptuf::Cb.fdh Vectors

DNA sequences of an fdh gene of Mycobacterium vaccae (Mv.fdh) and an fdh gene of Candida boidinii (Cb.fdh) were optimized to match codons of Corynebacterium glutamicum (SEQ ID NO: 3 and 4, respectively). To express the genes in the presence of tuf promoter of Corynebacterium glutamicum, the tuf promoter was cloned in BamHI and XhoI sites of the pGS_EX4 vector illustrated in FIG. 3 to obtain pGEX_Ptuf::Mv.fdh and pGEX_Ptuf::Cb.fdh vectors.

(2) Preparation of pGEX_P29::Mv.fdh and pGEX_P29::Cb.fdh Vectors

1) Preparation of a MD0375 Vector

A promoter of C. glutamicum NCgl1929 was PCR amplified by using a J0180 (SEQ ID NO: 46) and MD-1081 (SEQ ID NO: 47) primer set to obtain a 206 by PCR product, and inserted the 206 by PCR product into a pGST1 vector excised by KpnI and XhoI. Cloned vectors were introduced into One Shot TOP10 chemically competent cells (cat.# C4040-06, available from Invitrogen) and cultured in an LB culture medium including 25 mg/L of kanamycin. Vectors were recovered from the colonies formed, in order to confirm vector sequences through sequencing. The vector was named MD0375.

2) Preparation of pGEX P29::Mv.fdh and pGEX P29::Cb.fdh Vectors

An Mv.fdh gene (SEQ ID NO: 3) obtained from a genetic synthesis was used as a template and an Mv_fdh_{—}5′_F (SEQ ID NO: 48) and Mv_fdh_{—}3′_R (SEQ ID NO: 49) primer set was used for PCR amplification, and then the PCR product thereof were cloned in XhoI and BamHI restriction sites of an MD0375 vector to obtain a pGEX_P29::Mv.fdh vector.

A Cb.fdh gene (SEQ ID NO: 4) obtained through genetic synthesis was used as a template and a Cb_fdh_{—}5′_F (SEQ ID NO: 50) and Cb_fdh_{—}3′_R (SEQ ID NO: 51) primer set was used for PCR amplification, and the PCR product thereof were cloned in XhoI and BamHI restriction sites of an MD0375 vector to obtain pGEX_P29::Cb.fdh vector.

(3) Effects of Formate on Succinic Acid Production

pGEX_P29::Mv.fdh and pGEX_P29::Cb.fdh were each introduced into CGL (Δldh, ΔpoxB, Δpat-ackA, ΔactA), which is a parent strain of Example 1, to prepare an fdh gene expression strain. The expression strain was cultured under the same culturing conditions as in Example 4, except that formate was further added.

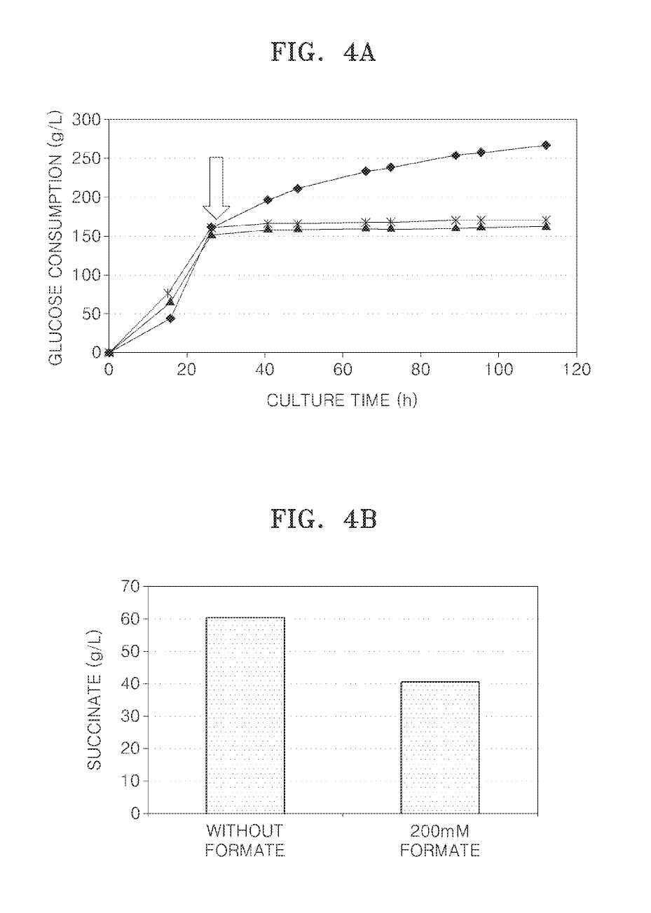

FIG. 4A shows changes in the amounts of glucose consumption of the fdh gene expression strain, after adding formate. ♦ represents changes in the amount of glucose consumption of a strain without the fdh gene. ▴ and x represent changes in the amounts of glucose consumption of the Mv.fdh gene and Cb.fdh gene expression strains, respectively. The arrow represents an addition of 200 mM of formate. The fdh gene expression strain stopped glucose consumption after the addition of formate.

Also, to observe the effects of formate on the production of succinic acid, a strain without the fdh gene was cultured under the same conditions as in Example 4.

FIG. 4B shows analysis results illustrating inhibitory effects of formate with respect to the production of succinic acid. The production of succinic acid was decreased by about 34% compared to the strain without formate. In order to resolve such problems, a method of increasing the production of succinic acid without the addition of formate was investigated.

### EXAMPLE 3

### Preparation of a Strain Including a Genome Integrated fdh Gene

To obtain a strain including a genome integrated fdh gene, a pK19ms_ΔpoxB_P29::Mv.fdh vector was prepared by the following method. Two homologous regions for the deletion of the poxB gene were PCR amplified by using genomic DNA of a C. glutamicum strain as a template and by using a poxB_up_NF (SEQ ID NO: 52) and poxB_up_NR (SEQ ID NO: 53) primer set, and a poxB_dn_NF (SEQ ID NO: 54) and poxB_dn_NR (SEQ ID NO: 55) primer set. P29::Mv.fdh region was PCR amplified by using a pGEX_P29::Mv.fdh vector as a template and by using a poxB_fdh_NF (SEQ ID NO: 56) and poxB_fdh_NR (SEQ ID NO: 57) primer set. Each DNA fragment was cloned in HindII and EcoRI restrictions sites of pK19 mobsacB vector and sequenced the same to analyze the sequences.

The pK19ms_ΔpoxB_P29::Mv.fdh vector was introduced into CGL (Δldh, ΔpoxB, Δpat-ackA, ΔactA) to prepare the strain including the genome integrated fdh gene, and confirmed whether the Mv.fdh gene was inserted into the ΔpoxB site by PCR amplification using poxB_C_F (SEQ ID NO: 58) and poxB_C_R (SEQ ID NO: 59) primer set.

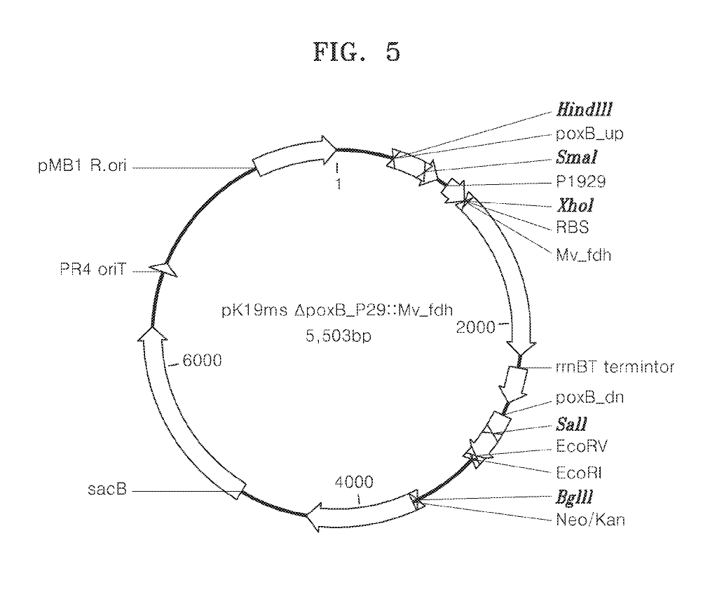

FIG. 5 is a map of the pK19ms_ΔpoxB_P29::Mv.fdh vector.

### EXAMPLE 4

### Analysis of the Succinic Acid Productivity of a Strain Including a Genome Integrated fdh Gene

The productivity of the strain including the genome integrated fdh gene prepared in Example 3 was compared to the productivity of a parent strain.

For a seed culture, each strain was streaked in an active plate including 5 g/L of yeast extract, 10 g/L of beef extract, 10 g/L of polypeptone, 5 g/L of NaCl, and 20 g/L of agar, and then the same was cultured at a temperature of 30° C. for 48 hours. A single colony was inoculated in 5 ml of a S1 culture medium including 40 g/L of glucose, 10 g/L of polypeptone, 5 g/L of yeast extract, 2 g/L of (NH₄)₂SO₄, 4 g/L of KH₂PO₄, 8 g/L of K₂HPO₄, 0.5 g/L of MgSO₄.7H₂O, 1 mg/L of thiamine-HCl, 0.1 mg/L of D-biotin, 2 mg/L of Ca-pantothenate, and 2 mg/L of nicotineamide, and then cultured the same at a temperature of 30° C. until an optical density at 600 nanometers value (“OD₆₀₀”) value reached 5.0. The culture medium was transferred to 70 ml of an S1 culture medium and then cultured at a temperature of 30° C. for 5 hours to prepare a seed culture medium.

The culturing began with 700 ml of the seed culture medium by using a 2.5 L fermenter and 5 mM of NH₄OH was used as a neutralizing agent. The seed culture medium was transferred to an SF1 culture medium including 150 g/L of glucose, 10 g/L of corn-steep liquor, 2 g/L of (NH₄)₂SO₄, 1 g/L of KH₂PO₄, 0.5 g/L of MgSO₄.7H₂O, 10 mg/L of FeSO₄.7H₂O, 10 mg/L of MnSO₄.H₂O, 0.1 mg/L of ZnSO₄.7H₂O, 0.1 mg/L of CuSO₄.5H₂O, 3 mg/L of thiamine-HCl, 0.3 mg/L of D-Biotin, 1 mg/L of calcium pantothenate, and 5 mg/L nicotinamide. The culture medium was cultured at a speed of 600 rpm and at 1.2 vvm until an OD₆₀₀ value of 120 was reached, and then cultured under the conditions of 200 rpm and 0 vvm.

After 134 hours of culturing under anaerobic conditions, a sample was collected and then centrifuged. The concentrations of succinic acid and glucose of a supernatant were analyzed through high performance liquid chromatography (HPLC).

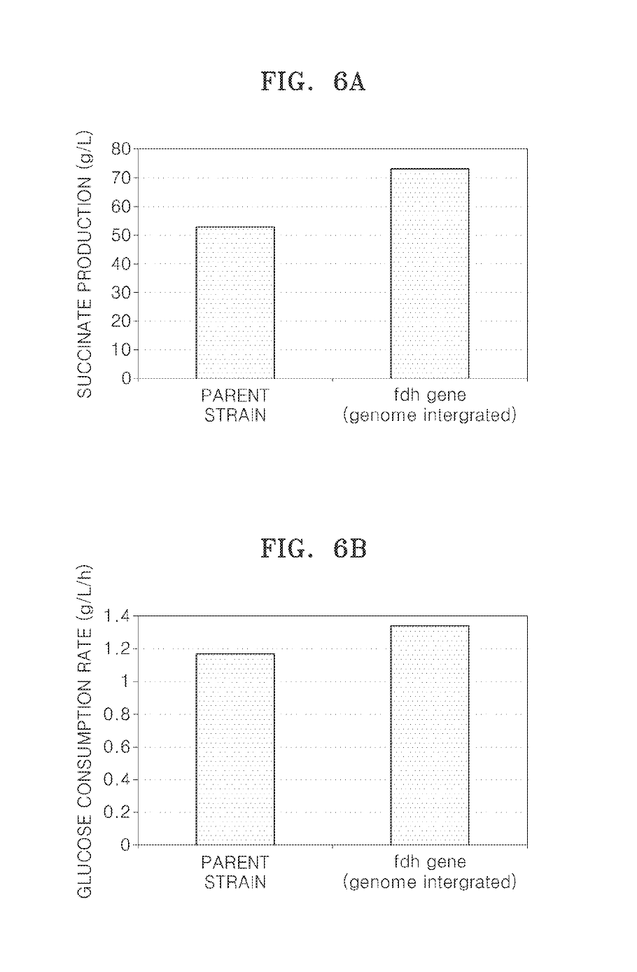

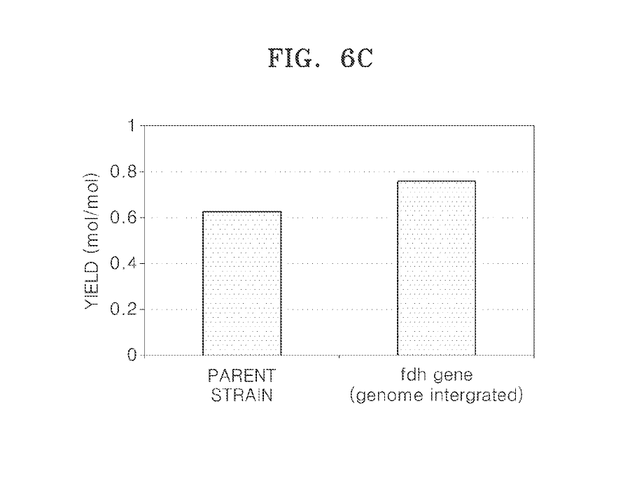

FIGS. 6A to 6C show results of culturing Corynebacterium in which an fdh gene has been integrated into the genome thereof. In FIG. 6A, a strain including a genomically inserted fdh gene showed productivity that is 27% greater than the productivity of a parent strain. In FIG. 6B, the strain showed a glucose consumption rate that is 12.5% greater than the glucose consumption rate of the parent strain. In FIG. 6C, the strain showed a succinic acid yield with respect to glucose that is 17.2% greater than that of the parent strain.

As described above, according to the one or more of the above embodiments of the present disclosure, a microorganism of Corynebacterium may be used for the production of reductive metabolites.

According other embodiments of the present disclosure, C4 dicarboxylic acid may be efficiently produced by using the method of producing C4 dicarboxylic acid.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant Corynebacterium microorganism comprising a gene that encodes NAD+ dependent formate dehydrogenase (FDH).

2. The recombinant Corynebacterium microorganism of claim 1, wherein the gene is from Mycobacterium vaccae or Candida boidinii.

3. The recombinant Corynebacterium microorganism of claim 1, wherein the NAD+ dependent formate dehydrogenase comprises SEQ ID NO: 1 or 2.

4. The recombinant Corynebacterium microorganism of claim 1, wherein the gene that encodes NAD+ dependent formate dehydrogenase comprises SEQ ID NO: 3 or 4.

5. The recombinant Corynebacterium microorganism of claim 1, wherein the gene is in a chromosome of the Corynebacterium microorganism.

6. The recombinant Corynebacterium microorganism of claim 1, wherein the recombinant Corynebacterium microorganism exhibits increased expression of NAD+ dependent formate dehydrogenase compared to a non-recombinant microorganism of the same type.

7. The recombinant Corynebacterium microorganism of claim 1, wherein the activity of at least one protein selected from the group consisting of lactate dehydrogenase (LDH), pyruvate oxidase (PoxB), phosphotransacetylase (PTA), acetate kinase (AckA), and acetate coenzyme A transferase (ActA), is eliminated or reduced in the recombinant Corynebacterium microorganism compared to a non-recombinant microorganism of the same type.

8. The microorganism of claim 7, wherein expression of at least one gene selected from the group consisting of a gene coding lactate dehydrogenase, a gene coding pyruvate oxidase, a gene coding phosphotransacetylase, a gene coding acetate kinase, and a gene coding acetate coenzyme A transferase is inactivated or attenuated in the recombinant Corynebacterium microorganism compared to a non-recombinant microorganism of the same type.

9. The microorganism of claim 1, wherein the recombinant Corynebacterium microorganism is a recombinant Corynebacterium glutamicum.

10. A method of producing a C4 dicarboxylic acid comprising: culturing the recombinant Corynebacterium microorganism of claim 1 to produce a cultured product comprising a C4 dicarboxylic acid; and recovering the C4 dicarboxylic acid from the cultured product.

11. The method of claim 10, wherein the recombinant Corynebacterium microorganism is cultured without an addition of formate.

12. The method of claim 10, wherein the recombinant Corynebacterium microorganism is cultured under microaerobic conditions or anaerobic conditions.

13. The method of claim 10, wherein the C4 dicarboxylic acid is succinic acid.

14. A method of producing a recombinant Corynebacterium microorganism comprising introducing into the Corynebacterium a gene encoding NAD+ dependent formate dehydrogenase (FDH).

15. The method of claim 14, wherein the introduction of the gene into the Corynebacterium is performed by homologous recombination.
